(19) United States
(12) Patent Application Publication
(10) Pub. No.: US 2014/0023624 A1
(43) Pub. Date: Jan. 23, 2014

(54) DECELLULARIZED LIVER TRANSPLANTATION COMPOSITION AND METHODS

(71) Applicants: Ping Zhou, Davis, CA (US); Jian Wu, Davis, CA (US); Jan Nolta, Davis, CA (US)

(72) Inventors: Ping Zhou, Davis, CA (US); Jian Wu, Davis, CA (US); Jan Nolta, Davis, CA (US)

(73) Assignee: The Regents of the University of California

(21) Appl. No.: 13/978,108

(22) PCT Filed: Dec. 30, 2011
(86) PCT No.: PCT/US11/68214

§ 371 Date: Sep. 24, 2013

Related U.S. Application Data
(60) Provisional application No. 61/429,430, filed on Jan. 3, 2011.

(51) Int. Cl. A61K 47/46; A61K 35/407; G01N 33/50

(52) U.S. Cl. CPC A61K 47/46; G01N 33/5008; A61K 35/407
USPC 424/93.7; 435/29

(57) ABSTRACT

This disclosure provides an isolated or purified decellularized liver extracellular matrix (DLM) composition containing an isolated or purified cell capable of differentiating into a hepatocyte and/or liver tissue, and methods for its use in vitro and in vivo.

### CROSS-REFERENCE TO RELATED APPLICATIONS

This application claims the benefit under 35 U.S.C. §119(e) of U.S. Provisional Application No. 61/429,430, filed on Jan. 3, 2011, the contents of which are hereby incorporated by reference in their entirety into the present disclosure.

### STATEMENT OF FEDERAL SUPPORT

This invention was supported by NIH grants: DK061848 and HL073256. The U.S. government has rights in this invention.

### BACKGROUND

Throughout this application, various patent and technical literature are referenced by an Arabic number. The complete bibliographic citation for these references are found immediately preceding the claims. These references, as well as the references cited within the subject specification, are incorporated by reference into this application.

Liver transplantation is the only established treatment for patients with acute liver failure, end-stage liver disease, and inherited liver-based metabolic disorders. However, the scarcity of donor livers means that many patients on the waiting list will never receive a liver transplantation and many more are never listed. The complexity of liver function makes it impossible to use only mechanical devices to provide temporary support, as has been employed for cardiac and renal failure. Extracorporeal liver support devices require viable hepatocytes for many functions; moreover, primary hepatocyte transplantation procedures cause less morbidity and mortality than whole organ transplantation, and may provide a sufficient cell mass to correct inherited metabolic deficiencies (1). Furthermore, we and others have demonstrated previously that transplantation of immortalized human fetal and neonatal hepatocytes in immunodeficient NOD-SCID mice via splenic injection allows the cells to migrate to the liver and mature in their liver-specific function (2, 3).

However, hepatocyte transplantation is still far from a routine practice in the treatment of liver diseases. For example, many hepatocytes die shortly after transplantation and the survival and proliferation rates of transplanted primary or fetal hepatocytes in experimental animal liver are often low even if prior liver injury was induced in the recipient mice (4). Additionally, only a limited number of hepatocytes or liver progenitor cells can be transplanted by the widely accepted methods of injection via the portal vein or spleen. Thus, transplanted cells are incapable of correcting any metabolic abnormalities or to rescuing fulminant liver failure unless they have a proliferative advantage over the recipient hepatocytes.

### SUMMARY

Transplantation of primary hepatocytes has been shown to augment the function of damaged liver and to “bridge” patients to liver transplantation. However, primary hepatocytes often have low levels of engraftment and short survival after transplantation. To explore the potential benefits of using decellularized liver extracellular matrix (DLM) as a carrier for hepatocyte transplantation, DLM from the whole mouse liver was generated. Immortalized human fetal hepatocytes (FH-hTERT) or primary human hepatocytes were infused into DLM, which was then implanted into the omentum of immuno-deficient NOD/SCID/IL2rγ−/− or NOD/SCID/MPS VII mice. The removal of endogenous cellular components and the preservation of the extracellular matrix proteins and vasculature were demonstrated in the resulting DLM. Bioluminescent imaging revealed that FH-hTERT transduced with a lentiviral vector expressing firefly luciferase survived in the DLM for 8 weeks after peritoneal implantation; whereas, the luciferase signal from FH-TERT rapidly declined in control mice 3-4 weeks after transplantation via splenic injection or with omental implantation after Matrigel encapsulation. Furthermore, primary human hepatocytes reconstituted in the DLM not only survived 6 weeks after transplantation, but also maintained their function, as demonstrated by mRNA levels of albumin and cytochrome P450 subtypes (CYP3A4, CYP2C9 and CYP1A1) similar to freshly isolated human primary hepatocytes. In contrast, when human primary hepatocytes were transplanted into mice via splenic injection, they failed to express CYP3A4, although they expressed albumin. In conclusion, decellularized liver extracellular matrix provides an excellent environment for long-term survival and maintenance of hepatocyte phenotype after transplantation.

This disclosure provides an isolated or purified decellularized liver extracellular matrix (DLM) composition comprising, or alternatively consisting essentially of, or yet further consisting of, an isolated or purified cell capable of differentiating into a hepatocyte and/or liver tissue and isolated or purified DLM. In one aspect, the composition comprises an amount of the cells capable of differentiating into hepatocytes, in an amount effective to support liver function when implanted into a patient. In another aspect, the effective amount is an amount to use for in vitro drug or biologic screening. In a further aspect, the composition further comprises, or alternatively consists essentially of, or yet further consists of a carrier such as a pharmaceutically acceptable carrier.

As used herein, an isolated or purified DLM intends a composition having no significant (e.g., less than 2%, or less than 4%, or less than 8%, or less than 10%, or less than 15%, or less than 20%) of cellular components. The removal of cellular components can be reflected by the color change of the liver during DLM preparation, e.g., semi-transparent. In one aspect, the isolated or purified DLM contains residual DNA content of less than 10%, or alternatively less than 8%, or alternatively less than 4%. The purified or isolated DLM comprises certain extracellular matrix (ECM) proteins, such as collagen IV, fibronectin and laminin, in the DLM. They can be verified by positive immunostaining of these ECM components. In one aspect and by way of example only, DLM can be prepared by cannulizing the portal vein as an inflow, and the inferior vena cava is cut as an opening of the outflow. Liver perfusion is carried out in situ at 37° C. and at the speed of 5 ml/minutes. Decellularization is achieved by sequential perfusion with, e.g., heparinized phosphate buffered saline, 1% sodium dodecyl sulfate (SDS) and 1% triton X. Detergents are washed away by perfusion with appropriate buffers and media. In a further aspect, the disclosure provides a method for preparing the composition by admixing a isolated or purified DLM with an effective amount of the isolate or purified cells. In one aspect, an effective amount is at least 500,00 cells, or alternatively at least 750,000 cells, or alternatively at least 1 million cells, or alternatively at least 1.25 million cells, or alternatively at least 1.5 million cells, or alternatively at least 2 million cells per 100 microliter of DLM or carrier.

In another aspect, the isolated or purified cell which is capable of differentiating into a hepatocyte and/or liver tissue is one or more of a hepatocyte precursor or stem cell, an embryonic stem cell or an induced pluripotent stem cell (iPSCs). In a further aspect, the composition further comprises, or alternatively consists essentially of, or yet further consists of, an isolated or purified mesenchymal stem cell.

The cell capable of differentiating into a hepatocyte and/or liver tissue and/or the isolated or purified DLM is not limited to a specific species, e.g., the cell and/or DLM is an animal or a mammalian origin. By way of example and without limitation, the mammalian cell is one or more of: a mouse cell, a rat cell, a simian cell, a canine cell, a porcine cell, a human cell, a bovine cell, an equine cell, a feline cell or an ovine cell.

The compositions can further comprise, or alternatively consist essentially of, or yet further consist of, of an effective amount of one or more of an isolated or purified hepatocyte, hepatocyte precursor cell, bone marrow, mesenchymal stem cell, umbilical cord blood-derived precursor endothelial cell, an endothelial cell isolated from placenta or other stem cell types.

The compositions as described herein are capable of maintaining liver function up to at least 6 weeks, or alternatively at least 8 weeks, or alternatively at least 10 weeks, or alternatively at least 12 weeks post transplantation in vivo.

This disclosure also provides the use of the above compositions for the preparation of a medicament. In one aspect, the composition is prepared with an effective amount of the cells capable of differentiating into an hepatocyte for an in vitro screen, or alternatively for an in vivo use as described herein. Drugs and biologics can be screen for possible effect on liver function, such as regeneration or supporting liver function.

This disclosure also provides a method for treating or preventing a disorder related to liver dysfunction comprising, or alternatively consisting essentially of, or yet further consisting of, administering to a subject in need thereof an effective amount of the compositions as described herein. In one aspect, the DLM composition is administered by implantation or injection into the omentum of the subject in need of such treatment.

In a further aspect, the disclosure provides a method for repairing or supporting liver function in a subject in need thereof, comprising, or alternatively consisting essentially of, or yet further consisting of, administering to a subject in need thereof an effective amount of the composition as described herein.

The above methods and uses can be further modified by co-administration (previous, subsequently concomitantly) of an effective amount of one or more of hepatocytes, hepatocyte precursor cells, mesenchymal stem cells, bone marrow or umbilical cord blood-derived precursor endothelial cells or endothelial cells isolated from placenta or other stem cell types to improve visualization of ischemic tissues (28-30). Thus, this disclosure also provides co-seeding hepatocytes with these cells in DLM to promote more rapid and robust revascularization. In another aspect, the method further comprises vessel anastomosis to the patient's systemic or portal circulation.

Further provided is a kit for in vitro or in vivo use as described herein comprising pre-prepared DLM and the cells, as well as instructions for use.

### BRIEF DESCRIPTION OF THE FIGURES

FIG. 1 Characterization of the decellularized liver matrix (DLM). (A) Representative mouse liver images of color changes during an in situ decellularization process at 0, 12, 30, 60 and 120 min of perfusion with 1% SDS. (B) DLM harvested from a mouse after the completion of a decellularization procedure. (C) H&E staining of a DLM slice demonstrating no remaining cellular components (100×). (D). DLM was injected with crystal violet in agarose through the portal vain after the completion of a decellularization procedure for the visualization of remaining vasculature networks (20×). (E) Mouse liver and the DLM cryosections were immuno-stained with antibodies against the indicated extracellular matrix proteins (fibronectin, laminin and collagen IV in green) and DAPI (blue) in the mouse liver. Please note that there was no DAPI staining in the DLM on the corresponding right panels (200×).

FIG. 2 FH-hTERT cultured in DLM. FH-hTERT transduced with a lentiviral vector carrying LUX-PGK-EGFP were infused into the DLM after the completion of perfusion (A) and cultured for 7 days (B & C). Fluorescent images were taken at 40× (A & B) and 200× (C) magnification. (D) Quantitative real-time RT-PCR analysis of ALB and AAT mRNA levels in FH-hTERT reconstituted in DLM cultured for 7 days. ** p<0.01 compared to FH-hTERT cultured in standard conditions (n=3).

FIG. 3 Bioluminescent imaging of FH-hTERT over time after transplantation. After transduction with lentiviral LUX-PGK-EGFP vector and enrichment by FACS, FH-hTERT were either infused into DLM and then implanted into mice or transplanted via splenic or omentum injection. (A) Representative bioluminescent images for the same mice over time with three modes of transplantation. (B) Bioluminescent signal intensity for the mice with splenic injection (n=5), omentum injection (n=4) or DLM implantation (n=4) at each time point. *, *** and **** correspond to P<0.05, 0.005 and 0.001 respectively in comparison to splenic injection at corresponding time points. A and AA correspond to p<0.05 and 0.01 in comparison to omentum injection at corresponding time points. The line indicates minimal signal strength to be imaged.

FIG. 4 DLM facilitates the survival of human primary hepatocytes in vivo. (A) GUSB staining (red) of human primary hepatocytes in the DLM 1 week after implantation into NOD/SCID/MPS VII mice. (B) Human primary hepatocytes transduced with the lentiviral LUX-PGK-EGFP vector and reconstituted in DLM were implanted into NOD/SCID/IL2rγ^{-/-} mice. The fluorescent image of the harvested DLM was made 6 weeks after implantation. GFP-positive human primary hepatocytes were visualized in green within the DLM.

FIG. 5 Quantitative real-time RT-PCR analysis of mRNA levels of the liver-specific gene: ALB (A), CYP3A4 (B), CYP1A1 (C) and CYP2C9 (D) in the livers or DLM implants of transplanted mice 6 weeks after transplantation. Human primary hepatocytes were either reconstituted in DLM or transplanted into in NOD/SCID/IL2rγ^{-/-} mice via splenic injection. The median value of each group is indicated with a bar. The number of animals from each group is shown in each plot, and there was no significant statistical difference in gene expression levels between DLM implantation and splenic injection in B, C and D. Expression levels of liver-specific genes were calculated based on that of freshly isolated human primary hepatocytes.

FIG. 6 Quantitative analysis of gene expression levels of hepatocyte-specific markers in hESC-derived hepatocytes cultured on DLM. ALB=human serum albumin; AAT=α1-antitrypsin; TAT=tyrosine amino transferase; TDO2=tryptophan 2,3-dioxygenase.

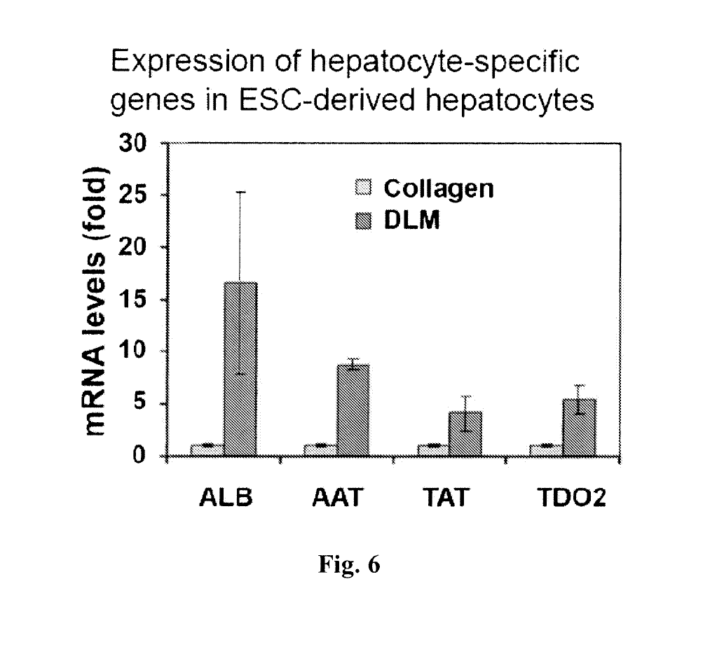

FIG. 7 Quantitative analysis of gene expression levels of hepatocyte-specific transcription factors in hESC-derived hepatocytes cultured on DLM. HNF1α=hepatocyte nuclear factor 1α; HNF4α=hepatocyte nuclear factor-4-α, C/EBPα=CCAAT enhancer binding protein alpha.

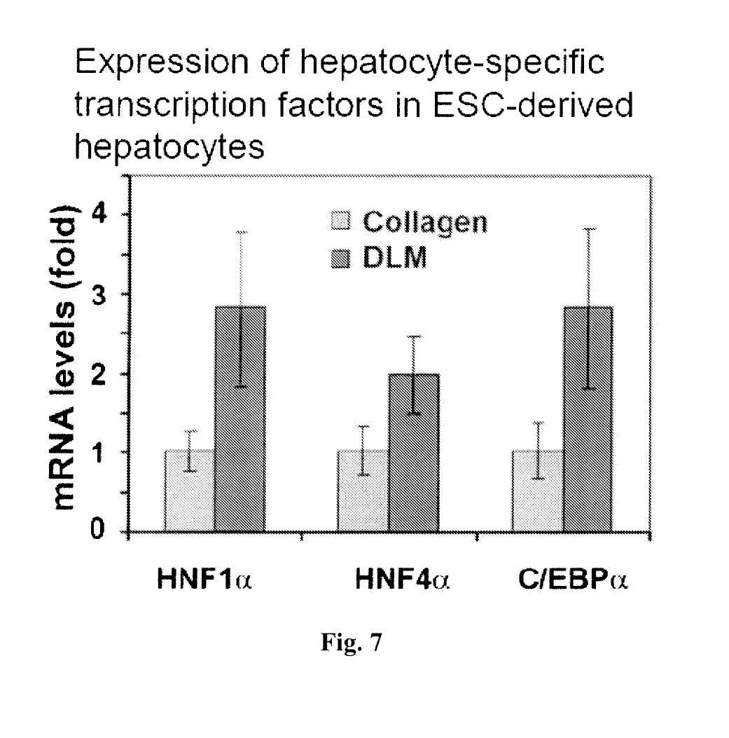

FIG. 8 Quantitative analysis of albumin levels in medium of ESC-derived hepatocytes cultured on DLM. Human primary hepatocytes were used as a positive control. Albumin levels were shown using total 10 μg RNA from cells in culture.

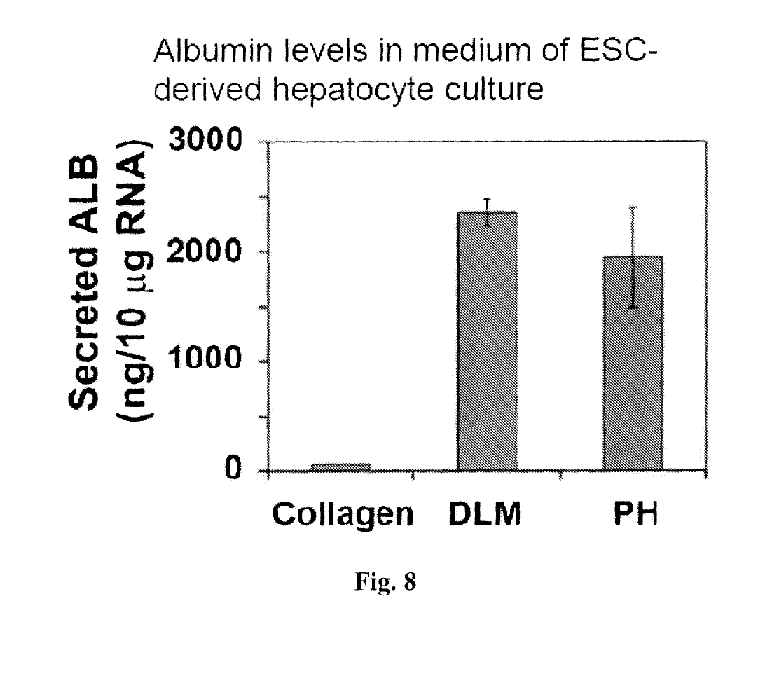

### DETAILED DESCRIPTION

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

A “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term “pharmaceutically acceptable carrier” (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

As used herein, the term “patient” or “subject” intends an animal, a mammal or yet further a human patient. For the purpose of illustration only, a mammal includes but is not limited to a human, a simian, a murine, a bovine, an equine, a porcine or an ovine.

As used herein, the term “oligonucleotide” or “polynucleotide” refers to a short polymer composed of deoxyribonucleotides, ribonucleotides or any combination thereof. Oligonucleotides are generally at least about 10, 15, 20, 25, 30, 40, 50, 60, 70, 80, 90, 100 or more nucleotides in length. An oligonucleotide may be used as a primer or as a probe.

The term “isolated” as used herein refers to molecules or biological or cellular materials being substantially free from other materials, e.g., greater than 70%, or 80%, or 85%, or 90%, or 95%, or 98%. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source and which allow the manipulation of the material to achieve results not achievable where present in its native or natural state, e.g., recombinant replication or manipulation by mutation. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides, e.g., with a purity greater than 70%, or 80%, or 85%, or 90%, or 95%, or 98%. The term “isolated” is also used herein to refer to cells or tissues that are isolated from other cells or tissues and is meant to encompass both cultured and engineered cells or tissues.

A “recombinant” nucleic acid refers an artificial nucleic acid that is created by combining two or more sequences that would not normally occur together. In one embodiment, it is created through the introduction of relevant DNA into an existing organismal DNA, such as the plasmids of bacteria, to code for or alter different traits for a specific purpose, such as antibiotic resistance. A “recombinant” polypeptide is a polypeptide that is derived from a recombinant nucleic acid.

As used herein, the term “promoter” refers to a nucleic acid sequence sufficient to direct transcription of a gene. Also included in the invention are those promoter elements which are sufficient to render promoter dependent gene expression controllable for cell type specific, tissue specific or inducible by external signals or agents.

In some embodiments, a promoter is an inducible promoter or a discrete promoter. Inducible promoters can be turned on by a chemical or a physical condition such as temperature or light. Examples of chemical promoters include, without limitation, alcohol-regulated, tetracycline-regulated, steroid-regulated, metal-regulated and pathogenesis-related promoters. Examples of discrete promoters can be found in, for examples, Wolfe et al. Molecular Endocrinology 16(3): 435-49.

As used herein, the term “regulatory element” refers to a nucleic acid sequence capable of modulating the transcription of a gene. Non-limiting examples of regulatory element include promoter, enhancer, silencer, poly-adenylation signal, transcription termination sequence. Regulatory element may be present 5′ or 3′ regions of the native gene, or within an intron.

Various proteins are also disclosed herein with their GenBank Accession Numbers for their human proteins and coding sequences. However, the proteins are not limited to human-derived proteins having the amino acid sequences represented by the disclosed GenBank Accession numbers, but may have an amino acid sequence derived from other animals, particularly, a warm-blooded animal (e.g., rat, guinea pig, mouse, chicken, rabbit, pig, sheep, cow, monkey, etc.).

As used herein, the term “treating” is meant administering a pharmaceutical composition for the purpose of improving the condition of a patient by reducing, alleviating, reversing, or preventing at least one adverse effect or symptom.

As used herein, the term “preventing” is meant identifying a subject (i.e., a patient) having an increased susceptibility to a disease but not yet exhibiting symptoms of the disease, and administering a therapy according to the principles of this disclosure. The preventive therapy is designed to reduce the likelihood that the susceptible subject will later become symptomatic or that the disease will be delay in onset or progress more slowly than it would in the absence of the preventive therapy. A subject may be identified as having an increased likelihood of developing the disease by any appropriate method including, for example, by identifying a family history of the disease or other degenerative brain disorder, or having one or more diagnostic markers indicative of disease or susceptibility to disease.

### Modes for Carrying Out the Disclosure

Primary hepatocytes lose their typical morphology and function in culture within a few days via dedifferentiation or epithelial mesenchymal transition (5, 6). This underscores the importance of the liver microenvironment in maintaining hepatocyte function. The extracellular matrix (ECM) not only provides a scaffold to house cells in liver tissue, but it also regulates adhesion, migration, differentiation, proliferation and survival of cells, as well as the interactions among different cell types (7). Recent advances in organ and tissue decellularization make it possible to obtain tissue-specific extracellular matrix from whole organs by perfusion of the organ with various detergents (8). Different from the traditional method of decellularization by immersing thin sliced tissues in various solutions for decellularization, the whole organ decellularized matrix maintains entire vascular network beds. These vascular network beds not only provide a convenient route for infusion of desired cell types but also a 3-dimensional environment for the infused cells in contrast to a 2-D environment provided from thin layers of decellularized matrix. Hence, we hypothesized that decellularized whole liver matrix (DLM) might provide an excellent microenvironment and scaffold for hepatocyte transplantation.

In the present disclosure, the feasibility and potential benefits of using decellularized liver extracellular matrix as a carrier for hepatocyte transplantation was explored. Whole mouse livers were decellularized and subsequently reconstituted with human primary hepatocytes or immortalized fetal hepatocytes (FH-hTERT). The resulting cell-reconstituted DLM scaffolds were implanted into the omentum of immuno-deficient mice. It was discovered that FH-hTERT survived longer when reconstituted in the DLM as compared to those that were directly transplanted into recipient mice via splenic injection or by omental implantation with Matrigel encapsulation. Primary human hepatocytes reconstituted in the DLM survived and maintained their liver-specific protein expression up to 6 weeks after the implantation of the DLM.

In one aspect, disclosed is a decellularized liver matrix (DLM) which is a natural scaffold of 3-dimensional extracellular matrix after removing all cellular components from a mammalian, e.g., mouse liver. The DLM is very useful for stem cell maturation and for the maintenance of differentiated function of epithelial cells, such as primary hepatocytes. The DLM were implanted after being reconstituted with either immortalized human fetal hepatocytes or human primary hepatocytes in immunodeficient mice. Immortalized fetal hepatoyctes survived one month more than other modes of cell transplantation, such as through splenic injection or injection directly into the omentum after extracellular matrix encapsulation. Primary hepacytes maintained liver-specific functions better when they were reconstituted in decellularized liver matrix than they were transplanted through splenic injection. Thud, this disclosure provides a method to generate a new liver or support a liver with stem cells, such as hepatocyte progenitor cells derived from embryonic stem cells or induced pluripotent stem cells, in decellularized liver matrix. This new liver can be implanted in recipients for a supporting therapy or for replacing a failing liver in patients with acute or chronic liver failure. There are needs for stem cell-engineered livers due to severe shortage of donor livers for end-stage of liver disease or fulminant liver failure. As compared to previously reported attempts for the use of recellularized liver matrix with rat liver cells, the previously reported attempts only survived up to 8 hours in rat recipients. In contrast, Applicants' DLM with human liver cells survived more than 2 months in mouse recipients.

In some embodiments, the present disclosure provides methods for preventing or treating liver disease in a patient, comprising administering to the patient an effective amount of an isolated decellularized matrix containing cells that can differentiate into liver tissue. In a particular aspect, the composition is administered to the patient in the omentum of the patient.

Any compositions described herein for a therapeutic use may be administered with an acceptable pharmaceutical carrier. Acceptable “pharmaceutical carriers” are well known to those of skill in the art and can include, but not be limited to any of the standard pharmaceutical carriers, such as phosphate buffered saline, water and emulsions, such as oil/water emulsions and various types of wetting agents.

As used herein, the term “administering” for in vivo and ex vivo purposes means providing the subject with an effective amount of the composition effective to achieve the desired object of the method. Methods of administering composition such as those described herein are well known to those of skill in the art and include, but are not limited to parenteral administration. The compositions are intended for topical, oral, or local administration as well as intravenously, subcutaneously, or intramuscularly. Administration can be effected continuously or intermittently throughout the course of treatment. Methods of determining the most effective means and dosage of administration are well known to those of skill in the art and will vary with the cell used for therapy, composition used for therapy, the purpose of the therapy, and the subject being treated. Single or multiple administrations can be carried out with the dose level and pattern being selected by the treating physician. For example, the compositions can be administered prior to or alternatively to a subject already suffering from a disease or condition that is linked to liver dysfunction.

As used herein, the term “sample” or “test sample” refers to any liquid or solid material containing nucleic acids or the compositions as described herein. In suitable embodiments, a test sample is obtained from a biological source (i.e., a “biological sample”), such as cells in culture or a tissue sample from an animal, most preferably, a human.

As used herein, the term “effective amount” refers to a quantity of a therapeutic composition delivered with sufficient frequency to provide a medical benefit to the patient.

A population of cells intends a collection of more than one cell that is identical (clonal) or non-identical in phenotype and/or genotype.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

As used herein, “stem cell” defines a cell with the ability to divide for indefinite periods in culture and give rise to specialized cells. Stem cells include, for example, somatic (adult) and embryonic stem cells. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell derived from the embryo that has the potential to become a wide variety of specialized cell types. An embryonic stem cell is one that has been cultured under in vitro conditions that allow proliferation without differentiation. Non-limiting examples of embryonic stem cells are the HES2 (also known as ES02) cell line available from ESI, Singapore and the H1 (also know as WA01) cell line available from WiCells, Madison, Wis. In addition, for example, there are 40 embryonic stem cell lines that are recently approved for use in NIH-funded research including CHB-1, CHB-2, CHB-3, CHB-4, CHB-5, CHB-6, CHB-8, CHB-9, CHB-10, CHB-11, CHB-12, RUES1, HUES1, HUES2, HUES3, HUES4, HUES5, HUES6, HUES7, HUES8, HUES9, HUES10, HUES11, HUES12, HUES13, HUES14, HUES15, HUES16, HUES17, HUES18, HUES19, HUES20, HUES21, HUES22, HUES23, HUES24, HUES26, HUES27, and HUES28. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4.

As used herein, a “pluripotent cell” broadly refers to stem cells with similar properties to embryonic stem cells with respect to the ability for self-renewal and pluripotentcy (i.e., the ability to differentiate into cells of multiple lineages). Pluripotent cells refer to cells both of embryonic and non-embryonic origin. For example, pluripotent cells includes Induced Pluripotent Stem Cells (iPSCs).

An “induced pluripotent stem cell” or “iPSC” or “iPS cell” refers to an artificially derived stem cell from a non-pluripotent cell, typically an adult somatic cell, produced by inducing expression of one or more reprogramming genes or corresponding proteins or RNAs. Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs and methods of preparing them are described in Takahashi et al. (2007) Cell 131(5):861-72; Takahashi & Yamanaka (2006) Cell 126:663-76; Okita et al. (2007) Nature 448:260-262; Yu et al. (2007) Science 318(5858):1917-20; and Nakagawa et al. (2008) Nat. Biotechnol. 26(1):101-6.

A “precursor” or “progenitor cell” intends to mean cells that have a capacity to differentiate into a specific type of cell such as a hepatocyte. A progenitor cell may be a stem cell. A progenitor cell may also be more specific than a stem cell. A progenitor cell may be unipotent or multipotent. Compared to adult stem cells, a progenitor cell may be in a later stage of cell differentiation.

The omentum (also known as the great omentum, omentum majus, gastrocolic omentum, epiploon, or, especially in animals, caul), is a large fold of visceral peritoneum that hangs down from the stomach. It extends from the greater curvature of the stomach, passing in front of the small intestines and reflects on itself to ascend to the transverse colon before reaching to the posterior abdominal wall.

### Compositions and Methods

In one aspect, described herein is an isolated or purified decellurarized liver extracellular matrix (DLM) composition comprising an isolated or purified cell capable of differentiating into a hepatocyte and/or liver tissue and isolated or purified DLM, in an effective amount. In one aspect, the composition further comprises an isolated or purified mesenchymal stem cell. In one embodiment, the composition can maintain liver function up to at least 6 weeks post transplantation in vivo. The DLM can be derived from any animal source, e.g. mammalian such as a mouse, a rat, a simian, a canine, a porcine, a human, a bovine, an equine, a feline or an ovine. The source can be the same as or different from the cell species. Although this disclosure describes the use of mouse DLM, it is apparent to those of skill in the art that the methods can be modified to any suitable animal source such as from organ donor tissue.

In one aspect, the cell capable of differentiating into a hepatocyte and/or liver tissue is selected from a hepatocyte precursor or stem cell, an embryonic stem cell or an induced pluripotent stem cell (iPSCs). The composition can further comprise an isolated or purified mesenchymal stem cell. In another aspect, the cells are animal cells, e.g., a mammalian cells. The cells can be autologous or allogeneic to the patient being treated and can be further modified to remove any potential for substantial graft versus host reaction upon transplantation or administration to the patient.

In one aspect, the mammalian cell is a mouse cell, a rat cell, a simian cell, a canine cell, a porcine cell, a human cell, a bovine cell, an equine cell, a feline cell or an ovine cell.

Various methods are provided. A method for treating or preventing a disorder related to liver dysfunction comprising administering to a subject in need thereof an effective amount of a composition as described herein. In another aspect, a method for repairing or supporting liver function in a subject in need thereof is disclosed, the method, comprising administering to the subject an effective amount of a composition f as described herein. A method for preparing a composition as described herein is provided by this disclosure. In one particular aspect, the subject is a human patient.

In another aspect of the disclosed methods, the cell in the composition is an animal cell, e.g., a mammal. In one aspect, the mammal is a mouse, a rat, a simian, a canine, a porcine, a human, a bovine, an equine, a feline or an ovine. The composition can be autologous or allogeneic to the subject being treated and can be further modified to remove any potential for substantial graft versus host reaction upon transplantation or administration to the subject.

Also disclosed herein is a method for screening a potential therapeutic agent for the ability to modulate liver function comprising contacting the potential therapeutic agent with an effective amount of the composition as disclosed herein, and monitoring the growth and differentiation of the cells, wherein a change in the growth or differentiation indicates the agent can modulate liver function and a lack in the change in the growth or differentiation indicates the agent can not modulate liver function.

In a further aspect, the method is modified by comprising comparing the growth or differentiation of the cell contacted with the agent with the growth and differentiation of a cell that is not contacted with the potential therapeutic agent.

In a further aspect, each of the above screening methods further comprise comparing the growth or differentiation of the cell with the growth or differentiation of a cell that has been contacted with an agent previously identified as modulating the growth or differentiation of the cell.

### Materials and Methods

### List of Abbreviations:

AAT, α1-antitrypsin; ALB, albumin; CYP, cytochrome p450 family; DAPI, 4,6-diaminidino-2-phenylindole; DLM, decellularized liver matrix; ECM, extracellular matrix; FH-hTERT, telomerase-immortalized human fetal hepatocytes; GUSB, beta-glucuronidase; NOD/SCID/IL2rγ^{-/-}, nonobese diabetic/severe combined immunodeficient/interleukin 2 receptor γ deficient; NOD/SCID/MPS VII, nonobese diabetic/severe combined immunodeficient/mucopolysaccharidosis type VII; hPH, human primary hepatocytes; RT-PCR, reverse transcriptase polymerase reaction. HNFα=hepatocyte nuclear factor-α; TAT=tyrosine amino transferase; TDO2=tryptophan 2,3-dioxygenase.

### Materials and Methods

### Cell Culture and Viral Transduction

The use of primary human hepatocytes and immortalized fetal hepatocytes was approved by the Institutional Review Board at the University of California, Davis, and was performed in accordance with the guidelines for the protection of human subjects. Human fetal hepatocytes (hFH) were procured by Prof. S. Gupta at Albert Einstein College of Medicine, Bronx, N.Y. with the approval of the Institutional Committee of Clinical Investigations. The immortalization of hFH by the reconstitution of the human telomerase gene was successfully achieved by ectopic expression of the telomerase reverse transcriptase using a retrovirus vector as we described previously (3). Immortalized FH-hTERT were cultured in DMEM high glucose (GIBCO) supplemented with 10% fetal bovine serum (FBS), 2 mM glutamine, 1% penicillin/streptomycin, 9×10⁻⁵ M insulin and 5×10⁻⁶ M hydrocortisone (Sigma-Aldrich Co. St. Louis, Mo.). Human primary hepatocytes (hPH) were isolated, plated into culture plates as previously described (9), and provided by the Liver Tissue Procurement and Distribution System (LTPADS). Culture medium was changed to complete HCM medium (Lonza, Walkersville, Md.) shortly after transfer by LPTADS (5). Cells were transduced with a lentiviral LUX-PGK-EGFP vector encoding the firefly luciferase and green fluorescent protein genes at a multiplicity of infection (MOI) of 20 in the presence of protamine sulfate (8 μg/ml) (4, 10). Seven days after transduction, GFP-positive FH-hTERT, but not hPH, were selected by fluorescence-activated cell sorting (FACS) as described previously (4).

Whole Liver Decellularization and Reconstitution of the Decellularized Liver Matrix with Hepatocytes.

All animal experiments were performed in compliance with the NIH Guidelines for experimental animals, and the animal protocol was approved by the Institutional Animal Care and Use Committee (IACUC). The liver perfusion procedure was performed according to a method previously described (11-13). Briefly, the portal vein was cannulated as an inflow, and the inferior vena cava was cut as an opening of the outflow. Liver perfusion was carried out in situ at 37° C. and at the speed of 5 ml/minutes. Decellularization was achieved by a method similar to the whole heart decellularization as described previously (8) with modifications. Briefly, mouse liver was perfused sequentially with heparinized phosphate buffered saline (PBS) (12.5 U heparin/ml) for 15 min, 1% sodium dodecyl sulfate (SDS) for 2 hrs and 1% Triton-X100 for 30 min. Detergents were washed away by perfusion with PBS for additional 3 hrs and medium without FBS for 10 min. In order to visualize the vascular networks, DLM was injected with crystal violet dissolved in 1% low melting agarose via the portal vain. Micrograph images of vasculature in the resulting DLM were taken under a microscope. In order to examine the efficiency of the decellularization procedure, both fresh mouse liver and DLM were minced. DNA content in the liver and DLM was extracted as previously described (14) and quantitated by a NanoDrop 2000 spectrophotometer (Thermo Scientific, Wilmington, Del.). To reconstitute the resulting DLM, FH-hTERT (2-4 million) or hPH (1-2 million) in 1 ml of medium were infused through a perfusion catheter after the completion of the decellularization procedure.

### Transplantation of Hepatocytes in Mouse Models

NOD/SCID/MPS VII mice (15) and NOD/SCID/IL2rγ^{-/-} mice (The Jackson Laboratories, Bar Harbor, Me.) were bred at the animal facility of the University of California, Davis. Mice that did not show thymoma or other tumor growth were included for data analysis. After culture for one day, decellularized liver matrix (approximately 0.5×0.5×0.1 cm in size) reconstituted with either FH-hTERT or hPH was implanted into the peritoneal cavity of immunodeficient mice by suturing the DLM into a pocket created by the omentum tissue. Animals were anesthetized with a mouse cocktail consisting of xylazine (5-10 mg/kg) and ketamine (50-100 mg/kg) in PBS by intraperitoneal injection. The middle incision was properly closed by silk suture. The first control group of animals was transplanted with one million human FH-hTERT or primary hepatocytes in 100 μl medium via splenic injection as as described in the art (4). The second control group received transplantation of FH-hTERT after Matrigel encapsulation (1 million cells in 100 μl of 25% Matrigel in medium (v/v)) into the omentum by direct injection.

### Immunohistochemical and Immunofluorescent Analysis

After decellularization or being harvested from implanted animals, DLM was frozen in optimal cutting temperature embedding medium (Sakura, Torrance, Calif.) and sectioned in 12 μm thickness. The DLM sections harvested from NOD/SCID/MPS VII mice were stained for β-glucuronidase (GUSB) activity as described previously (16). For immunostaining, frozen sections were fixed in 4% paraformaldehyde for 20 min, washed with PBS, and permeabilized with 0.2% Triton-X100 in PBS for 30 min. DLM sections were then blocked with 1% bovine serum albumin (BSA) for 1 hour and incubated with primary antibodies for 1-2 hrs. After washing with PBS, DLM sections were incubated with secondary antibodies conjugated with Alexa Fluor 488 (Invitrogen, Carlsbad, Calif.) for 1 hour. After washing with PBS, DLM sections were mounted with mounting medium containing 4,6-diaminidino-2-phenylindole (DAPI) (Vector Laboratories, Burlingame, Calif.). In order to examine cellular components in DLM sections, they were also stained for hematoxylin and eosin routinely. Primary antibodies against laminin and collagen IV were kindly provided by Dr. J. Peters (University of California, Davis), and were used at 1:400 dilution. Primary antibodies against fibronectin was obtained from Calbiochem (EMD, Gibbstown, N.J.), and used at 1:200 dilution.

### Quantitative Real-Time RT-PCR

Fresh mouse liver and recellularized DLM were mechanically minced. Total RNA was isolated using RNeasy kits (Qiagen, Valencia, Calif.). First strand cDNA was generated using reverse transcriptase (Applied Biosystems, Foster City, Calif.). cDNA was subsequently subjected to PCR amplification using the ABI 7300 system under default conditions (Applied Biosystems, CA). The primers and probes for the human serum albumin (ALB) and 1-antitrypsin (AAT) were described previously (3). The primers and probes for human glyceraldehyde-3-phosphate dehydrogenase (GAPDH), CYP3A4, CYP2C9 and CYP1A1 were purchased from Applied Biosystems. All samples were assayed in duplicate reactions and the means were normalized by the endogenous human GAPDH mRNA levels, and RNA levels were compared to RNA isolated from primary human hepatocytes right after receiving them from LTPADS, as described previously (3).

### Bioluminescent Imaging

Transplanted mice were injected intraperitoneally with D-luciferin potassium salt (150 mg/kg body weight in 100 μl PBS) and imaged under isofluorane anesthesia with the IVIS 100 Imaging System (Xenogen Corp.) at the Center for Molecular and Genomic Imaging, Department of Biomedical Engineering, UC Davis, for bioluminescent signals the day after transplantation and once a week thereafter (4). Individual mice were imaged for 5 min each time under anesthesia. Bioluminescence intensity was quantified in units of maximum photons per second per centimeter squared per steradian (p/s/cm²/sr) with the Living Imaging®2.50 software.

### Statistical Analysis

Bioluminescent intensity was expressed as means±SEM, and the data of splenic injection, omentum injection and DLM implantation were analyzed by the one way variance test, followed by Newman-Keuls test for multiple comparisons between any two groups at the corresponding time points. The in vitro RT-PCR data were analyzed by unpaired student t test. The in vivo RT-PCR data were expressed as a medium value, and the data comparing DLM implantation with splenic injection were analyzed by signed rank sum test. A p-value of less than 0.05 was considered as statistically significant.

### Results

### Decellularization of Mouse Liver

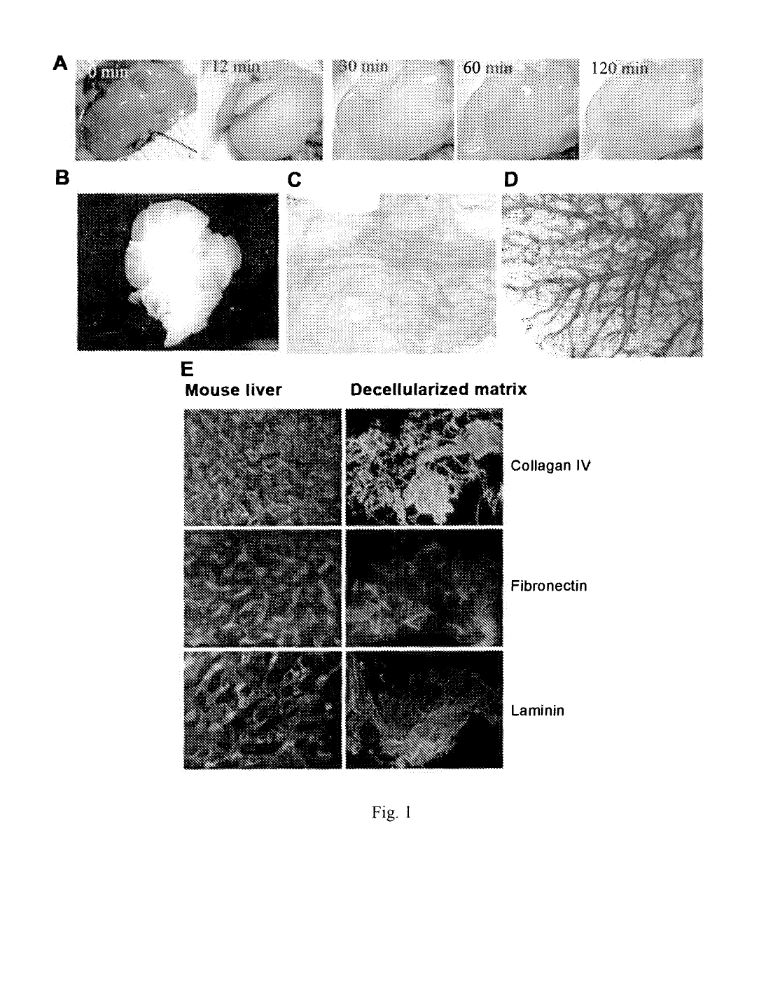

To create whole liver decellularized extracellular matrix, mouse liver was perfused in situ with a series of detergent solutions as previously described for rat heart decellularization. The removal of cellular components was reflected by the color change of the liver during the perfusion (FIG. 1A). The liver became semi-transparent after perfusion with 1% SDS for 2 hrs and then 1% Triton-X100 for 30 min (FIG. 1B). After subsequent perfusion with PBS for 3 hrs to wash away the remaining detergents, the resulting DLM was removed from the mouse, and cryopreserved and sectioned for further characterization. No significant remains of cellular components in the DLM were evidenced by H&E staining (FIG. 1C) and DAPI staining of these DLM sections (FIG. 1E). Residual DNA content in DLM was only 4% of the normal liver (73±39 μg/g DLM vs. 1750±291 μg/g liver). The vascular network was well preserved in DLM and was easily visualized by injection of crystal violet via the portal vein (FIG. 1D). The preservation of the extracellular matrix (ECM) proteins, such as collagen IV, fibronectin and laminin, in the DLM was verified by positive immunostaining of these ECM components (FIG. 1E). Therefore, this perfusion protocol with a series of detergent solutions effectively removed cellular components while preserving important extracellular matrix proteins, including collagen IV, fibronectin and laminin, as well as the vasculature.

### Survival of Immortalized Human Fetal Hepatocytes in DLM in Culture

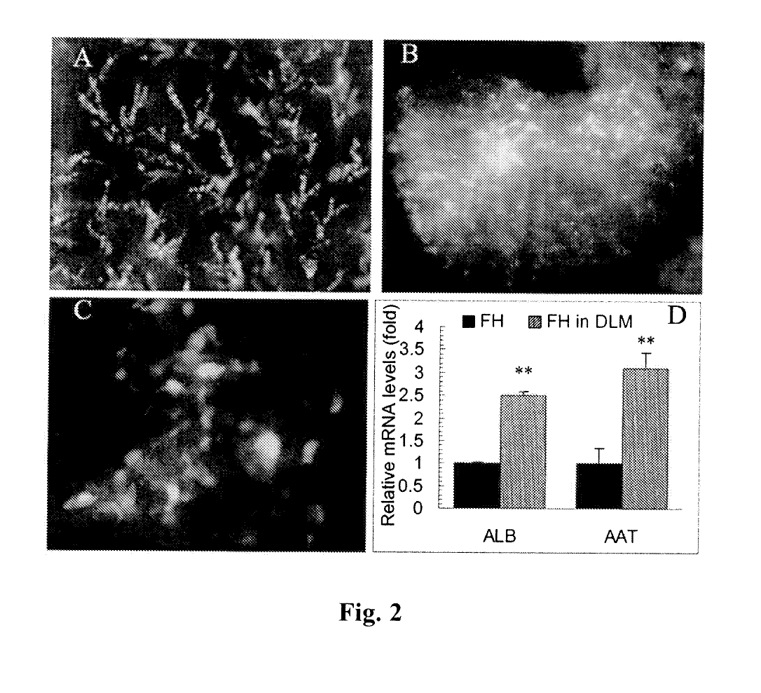

To assess whether the DLM facilitates the survival of liver cells, Applicants first used FH-hTERT transduced with a lentiviral LUX-PGK-EGFP vector encoding the luciferase gene and the green fluorescent protein (GFP) gene to reconstitute DLM via infusion. The majority of the cells remained within the vascular bed directly after the infusion (FIG. 2A). After culture for 1 week following cell reconstitution, GFP positive cells were still visible in the DLM and migrated into the parenchymal matrix (FIGS. 2B&C), suggesting that these reconstituted cells survived in the DLM. This was also shown using FH-hTERT without LUX-PGK-EGFP lentiviral transduction (data not shown). Furthermore, quantitative real-time RT-PCR analysis of human albumin (ALB) and α-antitypsin (AAT) mRNA levels in the DLM reconstituted with FH-hTERT showed a 2.5 to 3.5-fold increase in the levels of both hepatic-specific genes in comparison to FH-hTERT cultured under standard conditions (FIG. 2D), suggesting that these cells in DLM improved significantly in their hepatic-specific gene expression in vitro.

Bioluminescent Imaging of Mice Transplanted with FH-hTERT

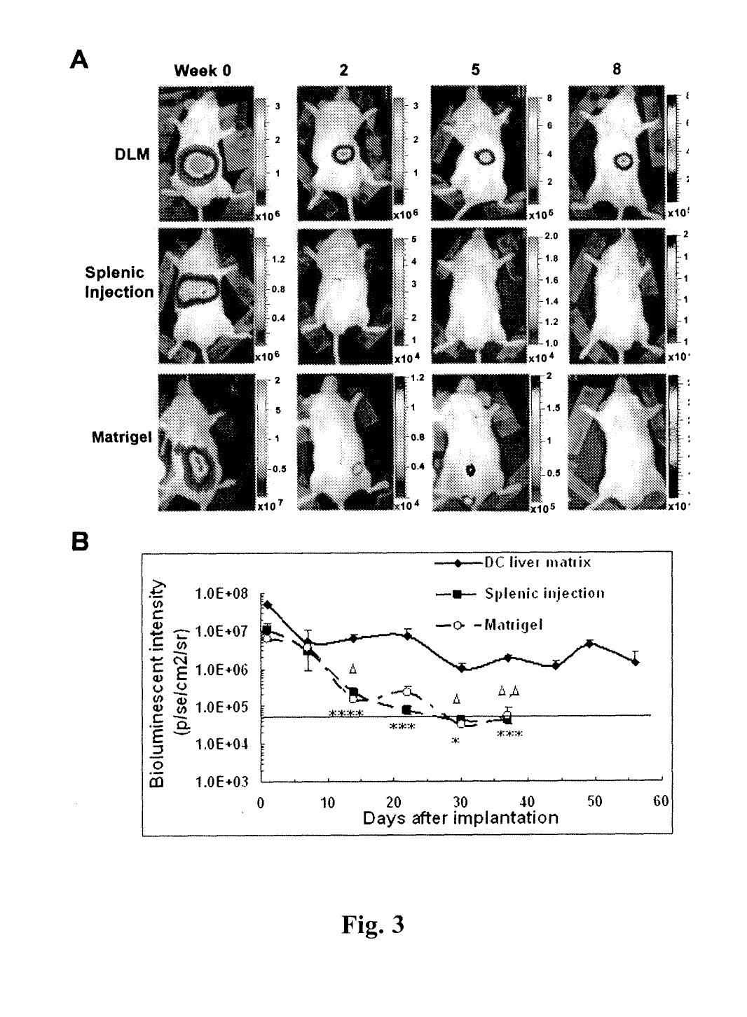

Having established that DLM supports the survival of FH-hTERT cells in vitro, Applicants next assessed whether the DLM facilitates the survival and function of these cells in vivo. The bioluminescent imaging modality offers a non-invasive approach to track the engraftment and repopulation of transplanted cells in vivo. To employ this technology, Applicants reconstituted DLM with FH-hTERT after transduction of the lentiviral LUX-PGK-EGFP vector, and then implanted the reconstituted DLM in the omentum of NOD/SCID/IL2rγ^{-/-} mice. For comparison, FH-hTERT with lentiviral vector transduction were injected into the spleen because splenic injection is a widely accepted method of hepatocyte transplantation in rodents. In a separate group, lentiviral vector-transduced FH-hTERT were first encapsulated in commercially available Matrigel, and then Matrigel-encapsulated FH-hTERT were injected into the omentum. Bioluminescent imaging of transplanted cells was conducted 1 day after cell transplantation, and once a week thereafter for 8 weeks. FIG. 3A shows repeated bioluminescent imaging of three representative mice at selected time points with DLM implantation, splenic or omentum injection; while FIG. 3B shows the average bioluminescent intensity of luciferase activity in these three groups of mice. Applicants found that bioluminescent signals rapidly faded in the liver area of mice with splenic injection within 3 weeks and that the bioluminescent signal strength declined to 0.39% of the initial level 37 days after splenic injection. The bioluminescent signal strength in mice receiving the injection of FH-hTERT with Matrigel encapsulation in the omentum declined (0.923%) in a trend similar to that of splenic injection. In contrast, bioluminescent signals declined less rapidly in mice transplanted with cells reconstituted in the DLM up to 8 weeks (2.65%), and statistically significant difference in bioluminescent intensity at several time points exists between the DLM group and the other 2 groups (p<0.05-0.001). These data clearly demonstrate that DLM enhanced the survival of immortalized fetal hepatocytes in vivo.

Survival of Human Primary Hepatocytes in DLM after Implantation

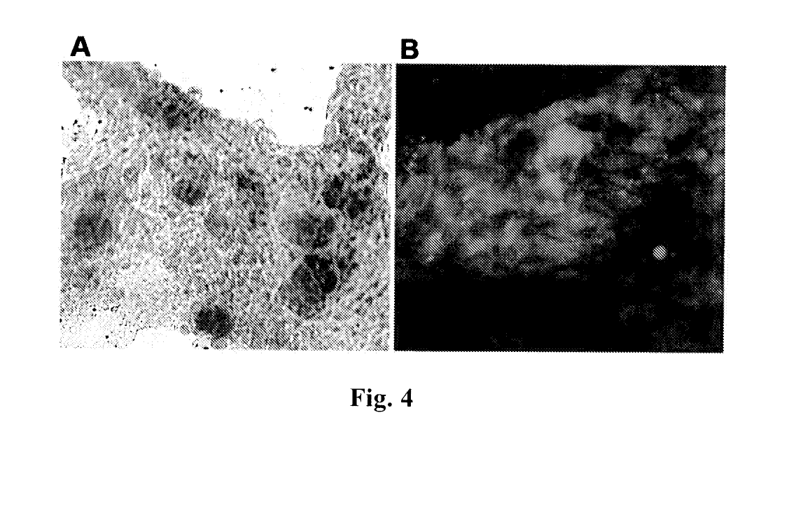

Applicants next assessed whether DLM is a good carrier for the transplantation of primary human hepatocytes. The DLM was reconstituted with hPH and the resulting scaffolds were implanted into the omentum of NOD/SCID/MPS VII mice. Since these mice were null for the enzyme of β-glucuronidase, which is encoded by the GUSB gene, human hepatocytes with normal GUSB expression can be easily visualized by using the substrate reaction to detect β-glucuronidase enzyme activity. One week after implantation, the implanted DLM was collected for β-glucuronidase staining. β-Glucuronidase-positive cells in red were clearly visible in the DLM (FIG. 4A). A similar experiment was performed using hPH transduced with the lentiviral LUX-PGK-EGFP vector in NOD/SCID/IL2rγ^{-/-} mice, a more severely immunodeficient strain. Six weeks after implantation, GFP-positive cells were identified in the DLM under a fluorescent microscope (FIG. 4B). It is also noticeable that GFP-negative mouse cells had migrated into the implanted DLM (FIG. 4B). Therefore, these data clearly demonstrate that the DLM facilitates the survival of human primary hepatocytes in vivo.

Function of Primary Human Hepatocytes in the DLM after Implantation

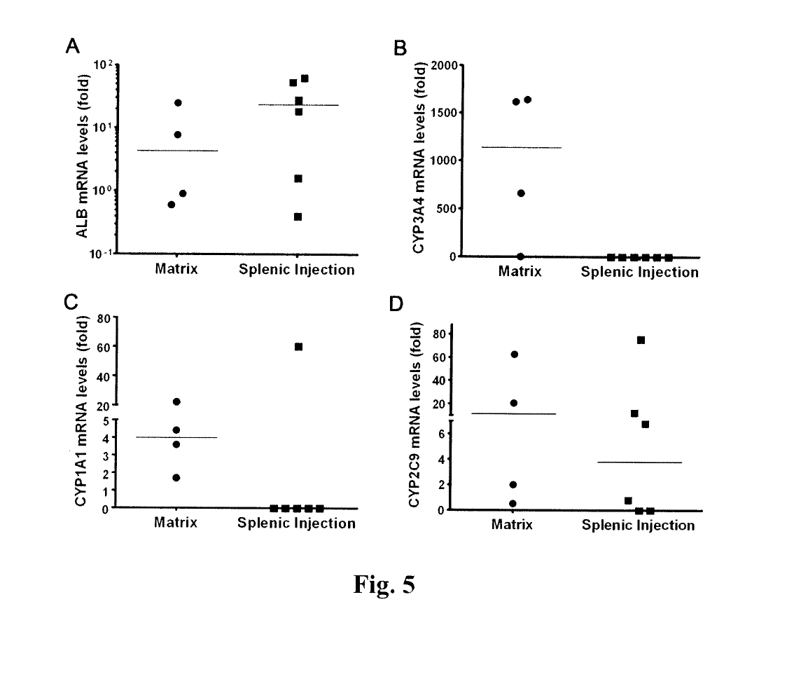

Having established that the DLM facilitates the survival of hPH, Applicants next examined whether hPH maintained their liver-specific function in DLM after being implanted into mice. Human primary hepatocytes were infused into DLM and subsequently the DLM reconstituted with human primary hepatocytes was implanted into the omentum of NOD/SCID/IL2rγ^{-/-} mice. Human primary hepatocyte transplantation via splenic injection was used as a control. Six weeks after implantation or transplantation, total RNA was isolated from the implanted DLM or the livers of the mice with splenic injection. Quantitative real-time RT-PCR analysis was carried out using RNA from freshly isolated hPH as a control to evaluate mRNA levels of the liver-specific genes in these samples. Cells in the DLM showed a level of albumin expression comparable to freshly isolated hPH (FIG. 5A). Human primary hepatocytes in mouse liver after splenic injection showed a similar level of albumin gene expression to cells in DLM (FIG. 5A), although their medium albumin expression level was slightly higher than cells in DLM (p>0.05). One of the hepatic-specific functions is to metabolize endogenous substrates and xenobiotics including drugs. The cytochrome P450 family enzymes (CYPs) catalyze the oxidation and transformation of endogenous or exogenous substances. CYP3A4 is the most abundant P450 subtype in the liver. Applicants found that hPHs reconstituted in DLM in 3 out of 4 mice exhibited a high level of CYP3A4 mRNA compared to the freshly isolated hPH (FIG. 5B). In contrast, hPHs after splenic injection did not show any CYP3A4 mRNA (FIG. 5B). Similarly, increased CYP1A1 expression was detected in hPHs reconstituted in DLM in all 4 mice, but it was absent in most of the mice (5 out of 6) with splenic injection (FIG. 5C). The CYP2C9 levels in hPHs reconstituted in DLM were similar to freshly isolated hPHs. hPHs transplanted in mice via splenic injection showed a detectable CYP2C9 mRNA level in 4 out of 6 mice (FIG. 5D). In summary, these data demonstrate that hPHs reconstituted in the DLM maintained liver-specific gene expression levels at least as high as splenic injection, and that two key markers of hepatocyte maturation, CYP3A4 and CYP1A1, were expressed at significantly higher levels in hPH that had been reconstituted in the decellularized matrix.

Comparison with Matrigel-Supported Stem Cells

Applicants modified a protocol published by Duan et al. (2007) Stem Cells 25(12):3058-3068, for hepatocyte differentiation from ESCs. The ESCs were first grown on Matrigel-coated plates using mouse embryonic fibroblast (MEFs)-conditioned ESC medium to reach around 70% confluence. Cells were then induced to differentiate to definitive endoderm by a sequential medium change to RPMI medium with activin A (100 ng/ml) for 24 h, to the same medium plus 0.5% fetal bovine serum (FBS) for 24 h and to RPMI medium with activin A (100 ng/ml), B27, and sodium butyrate (0.5 μM) for 4-6 days. Cells were lifted by trypsin treatment and plated into either collagen I-coated plates or decellularized liver matrices using the media described in Duan et al. (2007), supra, for 18-20 days. The culture medium was collected for analyzing the level of secreted human albumin by ELISA. The secretion of serum albumin is one of the main functions of mature hepatocytes. A robust increase in human albumin in the medium at a level comparable to primary human hepatocytes (PH) was observed in cells that were grown on DLM in comparison to those on collagen. Quantitative analysis of hepatocyte-specific gene levels in these cells revealed that DLM also significantly enhanced mRNA levels of hepatic markers, such as albumin (ALB), α1-antitrypsin (AAT), tyrosine amino transferase (TAT), and tryptophan 2,3-dioxygenase (TDO2) in comparison to those cultured on collagen. Furthermore, the mRNA levels of hepatic transcription factors, including HNF1α, HNF4α and C/EBPα, were also enhanced in cells grown on DLM compared to those on collagen. Based on these new data, Applicants conclude that DLM facilitated the further maturation of ESC-derived hepatocytes (ESC-Hep).

### Discussion

Decellularized extracellular matrix of blood vessels, cardiac valves, bladder and intestine has been used for facilitating cell transplantation (17-20). An in vitro study of using decellularized liver extracellular matrix for hepatocyte culture has been reported (21). It was shown that human hepatocytes cultured between two layers of porcine liver decellularized matrix in vitro for 10 days exhibited liver-specific function similar to those cells grown in a Matrigel sandwich (21), and that rat hepatocytes seeded between the sheets of decellularized liver matrix showed good viability and function in vitro (22, 23). Some of these previous studies employed pieces of decellularized liver matrices, and the decellularized matrix tissue was lyophilized into a powder form, and was rehydrated to generate a gel-like carrier. The data disclosed herein started with whole liver decellularization and cells that were infused into the DLM immediately after decellularization. This decellularization procedure which employed a much shorter period (6 hrs instead of 3 days) was as effective as a long decellularization protocol in terms of residual DNA content in the DLM (24). At the same time, the structure of DLM was extremely well preserved as demonstrated by full preservation of extracellular matrix and vasculature (FIG. 1). Moreover, the in vitro and in vivo data clearly demonstrated that the DLM facilitated both survival and function of human primary hepatocytes and fetal hepatocytes for up to 6-8 weeks after implantation as evidenced by bioluminescent imaging, immunohistochemical staining and quantitative RT-PCR assays.

Splenic injection has been widely used as a route for transplantation of hepatocytes in rodents (25). Cell survival between using the DLM as a carrier and splenic injection was compared, and it was found that fetal hepatocytes reconstituted in the DLM survived much longer than those with splenic injection. It appears that fetal hepatocytes migrated to the liver within a fewer days after splenic injection as demonstrated in our bioluminescent imaging study (data not shown). With this route of cell transplantation, the luciferase signal strength rapidly declined within 3 weeks after cell transplantation, which was similar to the findings previously reported when NOD-SCID mice were not pre-treated with methylcholanthrene and monocrotaline (4). An additional control group was added by the direct injection of HF-hTERT into the omentum after Matrigel encapsulation. The CCD camera imaging showed a trend of decline in bioluminescent intensity similar to that of splenic injection. In contrast, bioluminescent signal strength from HF-hTERT reconstituted into the DLM was sustained for up to 8 weeks. Presumably, the engraftment of HF-hTERT would be easier in DLM than in mouse liver because there is a vast space available, and intact extracellular matrix components in their original configuration remain after the completion of the decellularization. The result appeared to be better than when Matrigel was used to encapsulate HF-hTERT and encapsulated cells were implanted into the omentum (26). Human primary hepatocytes via either splenic injection or implantation in DLM survived in mice, and expressed liver-specific genes, such as albumin and CYP2C9. Moreover, primary hepatocytes in DLM expressed key mature markers, CYP3A4 and CYP1A1. This data indicate that DLM is superior to splenic injection for maintaining the function of primary human hepatocytes.

The establishment of a proper vascular system in the reconstituted DLM may be a critical issue for the survival of the transplanted cells. Bioluminescent imaging of FH-hTERT and primary hepatocytes with lentiviral LUX-PGK-EGFP transduction reconstituted in DLM revealed that the luciferase signals were sustained for a period of 8 weeks after implantation in NOD/SCID/IL2rγ^{-/-} mice, a strain of mouse which is to date the most immunodeficient, although the strength of the signals declined after the first week. These data indicate that the reconstituted cells may be able to access some, but not sufficient, blood supply as indicated by the presence of mouse cells in the implanted DLM. Applicants employed small pieces (0.5×0.5×0.1 cm³) of reconstituted DLM which were implanted in vascular-rich omentum in these experiments. This may have contributed to the prolonged survival and improved function of primary hepatocytes because the omentum has been a favorable site for engraftment of hepatocyte-polymer tissue-engineered constructs in comparison to subcutaneous compartments (26). However, when a larger size of DLM is needed for human cell transplantation, adequate blood supply with existing vasculature will be essential. Infusion of vascular endothelial cells or their precursor cells together with hepatocytes may facilitate the revascularization of the DLM. Linke et al. reported that pre-seeding a decellularized porcine jejunal segment with macrovascular endothelial cells before seeding porcine hepatocytes led to the maintenance of liver-specific function for 3 weeks in vitro (27). In previous studies, Applicants demonstrated that human bone marrow or umbilical cord blood-derived precursor endothelial cells or endothelial cells isolated from placenta and other stem cell types rapidly improved vascularization of ischemic tissues (28-30). Thus, this disclosure also provides co-seeding hepatocytes with these cells in DLM to promote more rapid and robust revascularization. Another modification of the methods comprise vessel anastomosis to the recipient's systemic or portal circulation (24). Although the recent study reported by Uygun et al. demonstrated the feasibility of the transplantation of a re-grown liver lobe from DLM with rat hepatocytes, the duration of the graft survival in rat recipients still requires improvement (24). In the present study Applicants have examined the long-term survival of human hepatocytes in an engineered liver graft.

The disclosed data suggest that DLM is an excellent carrier for transplantation of primary hepatocytes. However, the mechanism underlying this benefit is yet to be investigated. Integrins are major mediators of cell adhesion. ECM components including collagen and fibronectin bind to the RGD domain of integrins, and activate not only focal adhesion molecules but also cell survival signals, for instance, via the phosphoinositol-3, Akt or MAPK signaling pathways (31). In a study by Gupta and colleagues, infusion of collagen or fibronectin-like polymer through the portal vein prior to hepatocyte transplantation enhanced the engraftment of transplanted cells (32), which suggests a crucial role of extracellular matrix components in the integrity and function of transplanted hepatocytes. The decellularized liver matrix with the natural extracellular matrix components in a three-dimensional configuration appears to be responsible for prolonged survival and function of hepatocytes.

In conclusion, the findings in the present study demonstrate that decellularized liver matrix allows human fetal hepatocytes to survive longer than splenic or omentum injection in mice after transplantation. Moreover, the decellularized liver matrix maintains the liver-specific function of primary hepatocytes after implantation. Taken together, these data suggest the possibility that decellularized liver matrix may be developed as an alternative carrier for hepatocyte transplantation, when a large number of viable hepatocytes are required to functionally replace a failing liver.

In addition, a natural liver matrix carrier was created by removing all cellular components in mouse liver is provided. This decellularized liver matrix (DLM) does not possess any cellular components, but retains three dimensional structure of all extracellular matrix components in a perfect proportion with intact vessel structure, is an ideal natural microenvironment for mature hepatocytes or stem/progenitor cells for further differentiation or maturation in vitro or in vivo. The DLM was successfully reconstituted with either human fetal or primary hepatocytes and transplantion of the constructs in mice showed enhanced survival and fuction in comparason with the traditional splenic injection of hepatocytes. The recellularization of mature hepatocytes in DLM is highly useful in clinic, because DLM with mature hepatocytes is transplantable in patients with acute liver failure, end-stage of liver disorders or resection of liver malignancies as a bridge or substitution for orthotopic liver transplantation (OLT), which is the only established therapy for these illnesses. Due to severe shortage of donor livers, many patients with these illnesses on the waiting list will never have an opportunity to be transplanted. When DLM is used as a three dimensional microenvironment for the maturation or differentiation of stem/progenitor cells, such as embryonic stem cells (ESCs), or induced pluripotent stem cells (iPSCs), fetal hepatocytes or hepatoblast, etc. it should be more efficient and clinically relevant than other biological or synthetic matrices.

A series of detergents were used to flush out cellular components in mouse liver, and remaining is the architecture of extracellular matrices and vessel structure. The complete removal of cellular components was confirmed by no nucleus existence in the decellularized matrix. Immunohistochemical staining verifies the preservation of intact major extracellular matrices, such as collagen type IV, laminin and fibronection. After re-cellularization with either primary human hepatocytes or immortalized human fetal hepatocytes in DLM, these cells improved their hepatocyte-specific functions and protein production when they are cultured within DLM. Implantation of DLM after re-cellularization with immortalized human fetal hepatocytes in immuno-deficient mice extended the survival of these cells for more than one month, when compared to a standard method (splenic injection) of cell transplantation in mice. The living cells in implanted DLM were visualized by repeated bioluminescent imaging in recipient mice over two months. Moreover, when implantation of DLM after re-cellularization with primary human hepatocytes, these cells maintained a hepatocyte-specific gene expression profile superior to cells transplanted via splenic injection. These animal experiments have established the evidence of proof-of-concepts in the use of DLM as a carrier for hepatocyte transplantation, which has been less successful in clinic over past 30 years because of shortage of viable mature hepatocytes, the disorganized liver architecture after chronic injury (fibrosis/cirrhosis) and repopulation limit due to existence of host cells.

One possible source of DCM is cadaveric livers which are available when they are not suitable for transplant due to poor quality of donor livers or delayed time to collection resulting in cell death. The second alternative is to use normal livers from large animals, such as pigs. The genetic background of pigs is much more close to human than rodents, and the organ size is quite similar to human liver. After a complete removal of cellular components, there is reduced chance of xenogeneic infection, because most viruses live within cells. The only risk could be the potential immunologic incompatibility of extracellular matrices for humans. However, the antigenicity of foreign extracellular matrix components from a different species will be much less than a whole organ or cell components.

In one aspect, patient-specific iPSCs which do not possess any antigenicity to the same patient, and recellularize the DLM for his/her transplantation are generated. This approach would be relevant to conditions such as acute liver failure, complete removal of host liver due to trauma or malignancies, or end-stage of liver disorders as a result of cirrhosis, metabolic or genetic deficiencies. Now, it is possible to generate a large pool of iPSCs from nearly all genetic backgrounds, and these cells could in the future be used in major patients with various genetic background.

Due to its natural and three dimensional properties, have shown that DLM is the best microenvironment for the differentiation or maturation of stem/progenitor cells in vitro. A successful protocol of decellularization in the liver will be applicable in other organs, such as kidneys, lungs, heart, etc. and is a new technology for accelerated research in tissue engineering and organogenesis.

This liver used human fetal and adult hepatocytes to reconstitute murine decellularized liver tissue, which caused a longer and more durable graft and function than direct injection of the cell population.

This disclosure provides methods and compositions treat acute liver failure or end-stage liver diseases, presently, liver transplantation is the only established therapy. Due to the scarcity of the donor livers, only one fourth or fifth of patients eligible for the treatment will eventually receive a transplant, and many patients will die while waiting for donor organs. Moreover, many patients with severe liver disorders who otherwise can be treated by orthotopic liver transplantation (OLT) are not added into the waiting list largely due to the shortage of donor livers. The current alternative therapy for acute liver failure is to use an extracorporeal bioartificial liver device, which needs viable and functional hepatocytes to remove toxic substances, such as ammonia in the blood, and to substitute for critical protein synthesis. The second alternative is cell transplantation, which has not been fully successful after over 30 years of research due to the lack of viable mature hepatocytes, and disorganized architecture in chronic liver injury.

This disclosure also provides the use of decellularized liver matrix after recellularization with patient-specific iPSCs which are non-immunogenic to the recipient. The decellularized liver matrix (DLM) could be produced from cadaveric donor livers that are not suitable for transplant or from pig livers which have a large source.

Due to the fact that iPSCs are easily scaled up to a cell mass needed for detoxification and critical protein synthesis, there will be enough functional cell mass for recellularization in DLM.

DLM recellularized with iPSCs can be implanted in patients with liver failure. DLM is the best natural microenvironment for the maintenance of differentiated function and phenotypes of mature hepatocytes, and is superior to any artificial device in this aspect.

In contrast to cell transplantation in which transplanted cells will have less space in normal or damaged livers to survive and function, decellularized liver matrix provides a vast space in a natural three dimensional structure of extracellular matrix network and blood supply system once vascular endothelial cells are reconstituted. These neo-livers could also incorporate human mesenchymal stem cells which can form a support base for the hepatocytes and will rapidly enhance revascularization.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Fox I J, Chowdhury J R, Kaufman S S, Goertzen T C, Chowdhury N R, Warkentin P I, et al. Treatment of the Crigler-Najjar syndrome type I with hepatocyte transplantation. N Engl J Med 1998; 338:1422-6.
2. Wege H, Chui M S, Le H T, Strom S C, Zern M A. In vitro expansion of human hepatocytes is restricted by telomere-dependent replicative aging. Cell Transplant 2003; 12:897-906.
3. Wege H, Le H T, Chui M S, Liu L, Wu J, Giri R, et al. Telomerase reconstitution immortalizes human fetal hepatocytes without disrupting their differentiation potential. Gastroenterology 2003; 124:432-44.
4. Choi M S, Catana A M, Wu J, Kim Y S, Yoon S J, Borowsky A D, et al. Use of bioluminescent imaging to assay the transplantation of immortalized human fetal hepatocytes into mice. Cell Transplant 2008; 17:899-909.
5. Yamamoto N, Wu J, Zhang Y, Catana A M, Cai H, Strom S, et al. An optimal culture condition maintains human hepatocyte phenotype after long-term culture. Hepatol Res 2006; 35:169-77.
6. Copple B L. Hypoxia stimulates hepatocyte epithelial to mesenchymal transition by hypoxia-inducible factor and transforming growth factor-beta-dependent mechanisms. Liver Int 2010; 30:669-82
7. Svegliati-Baroni G, De Minicis S, Marzioni M. Hepatic fibrogenesis in response to chronic liver injury: novel insights on the role of cell-to-cell interaction and transition. Liver Int 2008; 28:1052-64.
8. Ott H C, Matthiesen T S, Goh S K, Black L D, Kren S M, Netoff T I, et al. Perfusion-decellularized matrix: using nature's platform to engineer a bioartificial heart. Nat Med 2008; 14:213-21.
9. Kostrubsky V E, Ramachandran V, Venkataramanan R, Dorko K, Esplen J E, Zhang S, et al. The use of human hepatocyte cultures to study the induction of cytochrome P-450. Drug Metab Dispos 1999; 27:887-94.
10. Oertel M, Rosencrantz R, Chen Y Q, Thota P N, Sandhu J S, Dabeva M D, et al. Repopulation of rat liver by fetal hepatoblasts and adult hepatocytes transduced ex vivo with lentiviral vectors. Hepatology 2003; 37:994-1005.
11. Wu J, Soderbergh H, Karlsson K, Danielsson A. Protective effect of S-adenosyl-L-methionine on bromobenzene- and D-galactosamine-induced toxicity to isolated rat hepatocytes. Hepatology 1996; 23:359-65.
12. Wu J, Liu S L, Zhu J L, Norton P A, Nojiri S, Hoek J B, et al. Roles of tissue transglutaminase in ethanol-induced inhibition of hepatocyte proliferation and alpha 1-adrenergic signal transduction. J Biol Chem 2000; 275:22213-9.
13. Zhang Y, Venugopal S K, He S, Liu P, Wu J, Zern M A. Ethanol induces apoptosis in hepatocytes by a pathway involving novel protein kinase C isoforms. Cell Signal 2007; 19:2339-50.
14. Laird P W, Zijderveld A, Linders K, Rudnicki M A, Jaenisch R, Berns A. Simplified mammalian DNA isolation procedure. Nucleic Acids Res 1991; 19:4293.
15. Zhou P, Hohm S, Olusanya Y, Hess D A, Nolta J. Human progenitor cells with high aldehyde dehydrogenase activity efficiently engraft into damaged liver in a novel model. Hepatology 2009; 49:1992-2000.
16. Hofling A A, Devine S, Vogler C, Sands M S. Human C D34+ hematopoietic progenitor cell-directed lentiviral-mediated gene therapy in a xenotransplantation model of lysosomal storage disease. Mol Ther 2004; 9:856-65.
17. Schmidt C E, Baier J M. Acellular vascular tissues: natural biomaterials for tissue repair and tissue engineering. Biomaterials 2000; 21:2215-31.
18. Schultheiss D, Gabouev A I, Cebotari S, Tudorache I, Walles T, Schlote N, et al. Biological vascularized matrix for bladder tissue engineering: matrix preparation, reseeding technique and short-term implantation in a porcine model. J Urol 2005; 173:276-80.
19. El-Hakim A, Marcovich R, Chiu K Y, Lee B R, Smith A D. First prize: ureteral segmental replacement revisited. J Endourol 2005; 19:1069-74.
20. Badylak S F, Park K, Peppas N, McCabe G, Yoder M. Marrow-derived cells populate scaffolds composed of xenogeneic extracellular matrix. Exp Hematol 2001; 29:1310-8.
21. Sellaro T L, Ranade A, Faulk D M, McCabe G P, Dorko K, Badylak S F, et al. Maintenance of human hepatocyte function in vitro by liver-derived extracellular matrix gels. Tissue Eng Part A 2009; 16:1075-82.
22. Burra P, Tomat S, Conconi M T, Macchi C, Russo F P, Parnigotto P P, et al. Acellular liver matrix improves the survival and functions of isolated rat hepatocytes cultured in vitro. Int J Mol Med 2004; 14:511-5.
23. Lin P, Chan W C, Badylak S F, Bhatia S N. Assessing porcine liver-derived biomatrix for hepatic tissue engineering. Tissue Eng 2004; 10:1046-53.
24. Uygun B E, Soto-Gutierrez A, Yagi H, Izamis M L, Guzzardi M A, Shulman C, et al. Organ reengineering through development of a transplantable recellularized liver graft using decellularized liver matrix. Nat Med 2010; 16:814-20.
25. Benten D, Kumaran V, Joseph B, Schattenberg J, Popov Y, Schuppan D, et al. Hepatocyte transplantation activates hepatic stellate cells with beneficial modulation of cell engraftment in the rat. Hepatology 2005; 42:1072-81.
26. Lee H, Cusick R A, Utsunomiya H, Ma P X, Langer R, Vacanti J P. Effect of implantation site on hepatocytes heterotopically transplanted on biodegradable polymer scaffolds. Tissue Eng 2003; 9:1227-32.
27. Linke K, Schanz J, Hansmann J, Walles T, Brunner H, Mertsching H. Engineered liver-like tissue on a capillarized matrix for applied research. Tissue Eng 2007; 13:2699-707.
28. Capoccia B J, Robson D L, Levac K D, Maxwell D J, Hohm S A, Neelamkavil M J, et al. Revascularization of ischemic limbs after transplantation of human bone marrow cells with high aldehyde dehydrogenase activity. Blood 2009; 113:5340-51.
29. Rosova I, Dao M, Capoccia B, Link D, Nolta J A. Hypoxic preconditioning results in increased motility and improved therapeutic potential of human mesenchymal stem cells. Stem Cells 2008; 26:2173-82.
30. Sondergaard C D H, Maxwell D, Weinheimer C, Rosová I, Creer M, Piwnica-Worms

D, et al. Human cord blood progenitors with high aldehyde dehydrogenase activity improve vascular density in a model of acute myocardial infarction. J Translat Med 2010: in press.
31. Scott M J, Billiar T R. Beta2-integrin-induced p38 MAPK activation is a key mediator in the CD14/TLR4/MD2-dependent uptake of lipopolysaccharide by hepatocytes. J Biol Chem 2008; 283:29433-46.
32. Kumaran V, Joseph B, Benten D, Gupta S. Integrin and extracellular matrix interactions regulate engraftment of transplanted hepatocytes in the rat liver. Gastroenterology 2005; 129:1643-53.

1.-7. (canceled)

8. A decellularized liver extracellular matrix (DLM) composition comprising an effective amount of an isolated or purified cell capable of differentiating into a hepatocyte and/or liver tissue and isolated or purified DLM.

9. The composition of claim 8, wherein the cell capable of differentiating into a hepatocyte and/or liver tissue is one or more of a hepatocyte precursor or stem cell, an embryonic stem cell or an induced pluripotent stem cell (iPSCs).

10. The composition of claim 8, wherein the composition further comprises an isolated or purified mesenchymal stem cell.

11. The composition of claim 8, wherein the cell is an animal cell or a mammalian cell, and wherein the mammalian cell is a mouse cell, a rat cell, a simian cell, a canine cell, a porcine cell, a human cell, a bovine cell, an equine cell, a feline cell or an ovine cell.

12. The composition of claim 8, wherein the effective amount is an amount that supports liver function when implanted into the omentum of a patient.

13. The composition of claim 8, wherein the composition maintained liver function up to at least 6 weeks post transplantation in vivo.

14. A method for treating or preventing a disorder related to liver dysfunction comprising administering to a subject in need thereof an effective amount of the composition of claim 8.

15. A method for repairing or supporting liver function in a subject in need thereof, comprising administering to a subject in need thereof an effective amount of the composition of claim 8.

16. The method of claim 14, wherein the composition is administered to the subject by implantation or injection into the omentum.

17. A method for screening a potential therapeutic agent for the ability to modulate liver function comprising contacting the potential therapeutic agent with an effective amount of the composition of claim 8, and monitoring the growth and differentiation of the cells, wherein a change in the growth or differentiation indicates the agent can modulate liver function and a lack in the change in the growth or differentiation indicates the agent cannot modulate liver function.

18. The method of claim 17, further comprising comparing the growth or differentiation of the cell contacted with the agent with the growth and differentiation of a cell that is not contacted with the potential therapeutic agent.

19. The method of claim 17, further comprising comparing the growth or differentiation of the cell with the growth or differentiation of a cell that has been contacted with an agent previously identified as modulating the growth or differentiation of the cell.
